(19) United States
(12) Patent Application Publication
(10) Pub. No.: US 2006/0116760 A1
(43) Pub. Date: Jun. 1, 2006

(54) Apparatus and method for treating presbyopia and other eye conditions

(75) Inventors: Spencer P. Thornton, Nashville, TN (US); James C. Hays, Atlanta, GA (US); Hideharu Fukasaku, Yokahama (JP)

Correspondence Address: DLA PIPER RUDNICK GRAY CARY US LLP, P. O. BOX 64807, CHICAGO, IL, 60664-0807, US

(73) Assignee: Human Technology Group, Inc.

(21) Appl. No.: 11/248,409

(22) Filed: Oct. 12, 2005

Related U.S. Application Data
(63) Continuation of application No. 10/999,567, filed on Nov. 30, 2004.

(51) Int. Cl. A61F 2/14; A61F 9/013

(52) U.S. Cl. 623/4.1; 128/898

(57) ABSTRACT

As humans age, there is a general loss of accommodation, termed presbyopia, which eventually leaves the eye unable to focus on near objects. This loss in ability to focus on near objects is a consequence of continued growth of the lens and crowding of the lens, zonules and ciliary body within the posterior chamber. Expansion of the posterior chamber is achieved by placing radial incisions in the sclera over the ciliary body. The method of correcting presbyopia according to the present invention is by making permanent the expansion of the sclera produced by incisions made in the sclera over the ciliary muscle by means of tissue barriers placed in the incisions, preventing contraction of the incision, and acting as a retainer of the increased circumference. With expansion of the posterior chamber, the effective working distance between the lens and ciliary muscle is restored and presbyopia reversed. The tissue barriers of the present invention are solid bars containing pre-placed integral sutures for fixation.

### RELATED APPLICATIONS

This Application is a continuation of U.S. application Ser. No. 10/999567, filed Nov. 30, 2004, and claims priority thereto.

### BACKGROUND OF THE INVENTION

1. Field of Invention

This invention relates to methods of treating presbyopia by means of surgical procedures and ophthalmic devices. This invention relates to increasing amplitude of accommodation of the eye by increasing the working space of the ciliary muscle-zonule-lens complex in the posterior chamber and allowing restoration of the hydrostatic and elastic forces within the eye. Primary open angle glaucoma and ocular hypertension are also treated by this invention.

2. Background

For near vision, the effective focal length of the human eye must be adjusted to keep the image of the object focused as sharply as possible on the retina. This change in effective focal length is known as accommodation. Accommodation, or the variation of focus of the eye on objects at varying distances, is thought to result from one or more of the following: 1) Thickening of the lens with zonular relaxation resulting in increased lens power; 2) Ciliary muscle contraction pulls vitreous forward, forcing the lens forward, with resulting power increase; and/or 3) Ciliary muscle contraction pulling on lens, thickening it centrally, resulting in power increase.

The posterior chamber of the eye contains the lens, zonules and ciliary muscle. The crystalline lens is ectodermal, just as is the skin, and continues to grow throughout life. Since the growing surface of the lens cannot be shed as skin is, the lens becomes larger as we age. The sclera, on the other hand, is mesodermal, as is bone and cartilage, and matures at puberty. Thus the sclera, or outer wall of the eye, does not grow after adulthood, and gradual crowding of the posterior chamber of the eye is the result.

Presbyopia is thought to be the result of aging of the eye with concomitant crowding of the posterior chamber. Because of this crowding with aging, there is decreased room for normal function of the ciliary muscle and the lens-zonule complex, and the result is presbyopia. Because of this continued growth of the lens within a non-growing chamber, crowding of the posterior chamber occurs with aging. Any procedure or device that could expand the space in the aging posterior chamber and restore normal function of the ciliary muscle/zonule/lens complex could therefore reverse presbyopia.

All incisions, unless tightly sutured or otherwise closed, act as if tissue is added. This is noted in the eye when radial incisions are made in the periphery of the cornea, as in radial keratotomy (RK). The spread of these radial incisions causes an increase in the circumference of the cornea at right angles to the direction of the incisions and therefore increase the corneal circumference in the area of the incisions. The result is an overall flattening of the cornea and a hyperopic shift in the focal power—a treatment for myopia or nearsightedness.

Expansion of the sclera is accomplished in a manner similar to RK by placing short radial incisions in the sclera over the ciliary body. The scleral incisions relax the sclera in the region of the ciliary body, resulting in expansion of the circumference of the eye and increasing the working distance between the ciliary body and the lens of the eye. Because this tissue is richly vascular and heals rapidly, the expansion produced by incisions may be rapidly lost without the aid of an artificial barrier to block the in-growth of new vessels and fibrosis and contraction of the incision.

One such procedure is known as anterior ciliary sclerotomy (ACS). ACS is a surgical incision technique used to correct up to about 2.00 diopters (D) of presbyopia. The procedure is based on the use of four or more radial incisions in the sclera over the ciliary body of the eye. The incisions expand the posterior chamber of the globe containing the ciliary body, zonules and crystalline lens, producing more room for the lens to accommodate for near vision. This effectively increases focal power and focal depth. The incisions are placed beginning at the surgical limbus, extending approximately 3 mm radially, stopping just anterior to the pars plana of the eye. This procedure can be enhanced by adding more incisions or reversed by suturing previously placed incisions.

One of the drawbacks of conventional ACS procedures is that the incisions rapidly heal and close, thereby reversing the beneficial effects of the procedure. Accordingly, a longer-lasting surgical method for increasing accommodation of the eye for near vision is needed. A barrier to prevent incision closure is therefore mandatory. Tissue barriers inserted in the radial incisions prevent closure and maintain the expansion produced by incisions. Maintaining the expansion produced by incisions is accomplished by inserting a barrier into the incision and fixing it permanently in place, resulting in an increase in the effective working distance of the ciliary muscle/lens/zonule complex in the posterior chamber of the eye.

A number of proposals have been made for changing the focal length of an implanted intraocular lens for cataract patients in response to the natural accommodation mechanism of the eye, such as in U.S. Pat. No. 4,254,509 to Tennant. One type of adaptive lens comprises an artificial lens whose shape is changed in response to the contraction and expansion of the ciliary muscle. This type of lens is an eye implant that has an optical lens anteriorly convex and posteriorly planar supported on two diametrically opposed coplanar feet through two supporting members forming an arch. Each supporting member is unitary with the lens and rooted in one of the feet outside the perimeter of the lens to support the lens with the posterior thereof anterior to the plane of the feet. The lens is formed of a rigid biologically inert material. The supporting members are formed of soft biologically supporting material. The resulting structure, when fixed into the sclera of the eye, will change as to move the lens anteriorly when forces are applied to the feet upon contraction of the ciliary body.

U.S. Pat. No. 4,718,904 to Thornton, discloses how ciliary muscle contraction moves the lens forward in accommodation by pulling the vitreous forward—showing that anterior movement of the lens is an integral component of accommodation. This is accomplished by providing more space in the posterior chamber of the eye. The proofs were referenced in the peer-reviewed publication Current Canadian Ophthalmic Practice, June 1986, “Lens Implantation With Restored Accommodation”. Loss of accommodation is treated by a method that increases the amplitude of accommodation by increasing the effective working distance of the ciliary muscle in the presbyopic or cataractous eye with an intraocular lens that takes up less space in the posterior chamber of the eye than the natural lens, thus effectively increasing the effect of ciliary muscle function. Similar ideas have been disclosed by U.S. Pat. No. 4,842,601 to Smith, U.S. Pat. No. 4,888,012 to Horn, et al., and U.S. Pat. No. 4,253,199 to Banko. Two other types of adaptive lenses are described in U.S. Pat. No. 4,994,082 to Richards, et al., and U.S. Pat. No. 5,275,623 to Sarfarazi shows a similar type of compound adaptive lens.

U.S. Pat. No. 4,790,847 to Woods, U.S. Pat. No. 5,152,789 to Willis, and U.S. Pat. No. 4,409,691 to Levy describes adaptive lens systems utilizing a simple intraocular lens. These systems have focusing capabilities that are achieved by axially shifting the lens in response to normal contraction and expansion of the ciliary muscle resulting from changes in range between the eye and an object under observation. These patents describe similar systems for providing motion of the lens. In each case the ciliary muscle controls zonules, which in turn provide tension to a lens capsule in which the lens system is mounted. The extremities of the capsule press against a radially compelled spring-like structure, which also forms a relatively large angle of somewhat less than 90 degree with the optical axis of the eye. The lens is positioned on the optical axis. Relaxation of the ciliary muscle releases the radial force and allows the spring to form a more nearly flat shape. When the ciliary muscle contracts, the pressure on the spring is increased by the action of the lens capsule, the angle between the spring and the optical axis is decreased, and the lens moves axially away from the ciliary muscle.

U.S. Pat. No. 5,047,051 to Cumming discloses an accommodative lens system utilizing a lens with hinged haptics that focus the lens by axially moving forward with accommodation. When the ciliary muscle contracts, the pressure of the vitreous pushes the lens forward, effectively increasing its focal power.

U.S. Pat. No. 5,354,331 to Schachar, discloses how presbyopia and hyperopia are treated by a method that increases the amplitude of accommodation by increasing the effective working distance of the ciliary muscle in the presbyopic eye. This is accomplished by expanding the sclera in the region of the ciliary body. A relatively rigid band having a diameter slightly greater than that of the sclera in that region is sutured to the sclera in the region of the ciliary body. The scleral expansion band comprises anterior and posterior rims and a web extending between the rims, the anterior rim having a smaller diameter than the posterior rim.

U.S. Pat. No. 5,465,737 to Schachar. The disclosures are similar to those of the '331 patent, except that the expansion of the sclera is accomplished by suturing to the sclera a relatively rigid band having a diameter slightly greater than that of the sclera in the region of the ciliary body, by relaxing the sclera overlying the ciliary body, by surgical procedures or treatment with enzymes, heat or radiation, whereby intraocular pressure expands the relaxed sclera, or by surgical alloplasty. The effective working distance of the ciliary muscle can also be increased by shortening the zonules by application of heat or radiation, by repositioning one or both insertions of the ciliary muscle or by shortening the ciliary muscle. Presbyopia is also arrested according to the invention by inhibiting the continued growth of the crystalline lens by application of heat, radiation or antimitotic drugs to the epithelium of the lens. Primary open angle glaucoma and/or ocular hypertension can be prevented and/or treated by increasing the effective working range of the ciliary muscle according to the invention.

U.S. Pat. Nos. 5,489,299; 5,722,952; 5,503,165, and 5,529,076 to Schachar contain essentially the same ideas as U.S. Pat. Nos. 5,354,331 and 5,465,737 with some improvements such that presbyopia and hyperopia are treated by a method that increases the amplitude of accommodation by increasing the effective working distance of the ciliary muscle in the presbyopic eye by shortening the zonules by application of heat or radiation, by repositioning one or both insertions of the ciliary muscle or by shortening the ciliary muscle. Presbyopia is also arrested by inhibiting the continued growth of the crystalline lens by application of heat, radiation or antimitotic drugs to the epithelium of the lens. Primary open angle glaucoma and/or ocular hypertension can be prevented and/or treated by increasing the effective working range of the ciliary muscle.

U.S. Pat. No. 6,007,578 to Schachar discloses how presbyopia is treated by implanting within a plurality of elongated pockets formed in the tissue of the sclera of the eye, a prosthesis having an elongated base member having an inward surface adapted to be placed against the inward wall of the pocket, and having a ridge on the inward surface of the base extending along at least a major portion of the major dimension of the base. By making a small incision in the outer coat of the eye (the sclera) and tunneling around the sclera over the ciliary body, one can place a polymethylmethacrylate (PMMA) arched stent within this scleral tunnel that will exert a stretching effect on the equatorial lens fibers. Four of these are placed around the eye in the sclera (approximately 2 mm away from the cornea), and approximately 90 degrees apart. From the Schachar hypothesis of accommodation it could be deduced that if one were able to increase the distance between the lens equator and the ciliary muscle, one could again stretch the zonules and reverse the effect of lens growth and to narrow this distance. The combined effect of the implanted prostheses is to exert a radially outward traction on the sclera in the region overlying the ciliary body, which expands the sclera in the affected region together with the underlying ciliary body to restore the effective working distance of the ciliary muscle in the presbyopic eye and thereby increases the amplitude of accommodation.

UK Pat. No, 2394899 to Hays, and UK Pat. No. 2394900 to Waldock and Hays disclose a method of preventing contraction of scleral incisions by inserting titanium tissue barriers in the incisions. The titanium barriers act as spacers, and are held in the incisions by means of projections from the sides or the ends of the barriers.

U.S. Pat. No. 6,006,756 to Shadduck, discloses a system and technique called magnetoresonant induction of an intrastromal implant that is adapted for corneal re-shaping. The technique is utilized to correct mild to high hyperopia and presbyopia by steepening the anterior corneal curvature in a single treatment, or in periodic treatments over the lifetime of the patient. The system comprises a combination of components including (i) at least one implantable magnetoresonant intrastromal segment, and (ii) an oscillating magnetic field generator together with a dosimetry control system including at least one emitter body adapted for positioning proximate to the patient's eye and intrastromal implant. The system can deliver thermal effects to appropriate stromal lamellae by non-contact inductive heating of the implant that in turn contracts or compresses stromal collagen fibrils into a circumferential cinch about an anterior layer of the cornea and steepens the anterior corneal curvature. A dosimetry control system controls the power level and duration of exposure of the oscillating magnetic field(s) and may be combined with intraoperative corneal topography.

U.S. Pat. No. 5,782,894 to Israel, discloses a device and method for treating presbyopia by which the ciliary muscles of the eyes are electrically stimulated when the internal rectus muscles of the eyes are activated in order to focus the eyes on objects within the near field of vision. The amounts of electrical stimulation can be adjusted according to the individual needs of a patient and are preferably in direct proportion to the amounts of contraction of the internal muscles.

U.S. Pat. No. 4,961,744 to Kilmer, et al, discloses a surgical apparatus for inserting a plastic, split end, adjusting ring into the stroma of the cornea of the eye wherein the adjusting ring includes, as a part thereof, a dissecting head to part the stroma and provide a pathway for the adjusting ring as the ring is rotated. The ends of the adjusting ring are moved to change the shape of the cornea to a desired shape in accordance with the desired visual correction after which the ends of the adjusting ring are fixably joined to maintain the desired shape.

U.S. Pat. No. 5,300,118 to Silvestrini, et al, discloses an intrastromal corneal ring (ICR) that is adjustable in thickness and has an elongated, flexible, preferably transparent or translucent body that forms a circle. The ICR is of a size such that it can be inserted into a human eye and is comprised of a material that is compatible with human ocular tissue.. A plurality of different embodiments of ICRs are disclosed each of which are adjustable in terms of their thickness. The thickness may be adjusted prior to the insertion of the ICR into the cornea and may not be further adjustable after insertion. However, in accordance with preferred embodiments, the ICR is inserted at a thickness that is believed to be proper and may thereafter be further adjusted in order to precisely define the desired thickness and thereby more precisely adjust the shape of the cornea, and focus the light entering the eye on the retina.

U.S. Pat. No. 5,824,086 to Silvestrini, discloses a pre-formed intrastromal corneal insert. It is made of a physiologically compatible polymer and may be used to adjust corneal curvature and thereby correct vision abnormalities. The insert or segment may also be used to deliver therapeutic or diagnostic agents to the interior of the cornea or of the eye. The insert subtends only a portion of a ring or “arc” encircling the anterior cornea outside of the cornea's field of view. The invention also includes a procedure for inserting the device into the cornea.

U.S. Pat. No. 6,051,023 to Kilmer, et al, discloses a surgical apparatus for inserting a plastic, split end, adjusting ring into the stroma of the cornea of the eye wherein the adjusting ring includes, as a part thereof, a dissecting head to part the stroma and provide a pathway for the adjusting ring as the ring is rotated. The ends of the adjusting ring are moved to change the shape of the cornea to a desired shape in accordance with the desired visual correction after which the ends of the adjusting ring are fixably joined to maintain the desired shape.

U.S. Pat. No. 5,888,243 to Silverstrini, discloses an intrastromal corneal ring housing comprising at least one outer layer of a physiologically compatible polymer having a low modulus of elasticity, which polymer may be hydratable and may be hydrophilic. The inner portion of the hybrid intrastromal corneal ring may be hollow or may contain one or more physiologically compatible polymers.

It is clear from the above cited patent literature search that no prior art has considered the possibility of accommodating the natural eye lens or intraocular lens with radial incisions beginning at the limbus and carried over the ciliary body in the sclera, with the expansion produced by the incisions maintained by a tissue barrier consisting of a rod of silicone or other inert material fixed into the incisions with integral pre-placed sutures to restore natural eye lens accommodation by the contraction of ciliary muscle in near vision situations.

Accordingly, it is an object of this invention to provide a method for treating presbyopia by increasing the radial distance between the lens and the ciliary muscle in the presbyopic eye.

### SUMMARY OF INVENTION

An apparatus and method for treating presbyopia and other eye disorders has now been found which comprises enlarging the posterior chamber and increasing the effective working distance of the ciliary muscle in the presbyopic eye, as further described herein. The present invention retains the effect of radial incisions with resulting restoration of ciliary body-zonule-lens relationships allowing changes in lens morphology and forward movement of the lens on accommodation. The method of this invention is an expansion barrier of silicone or other solid material inserted into incisions made in the sclera over the ciliary body.

### BRIEF DESCRIPTION OF THE DRAWINGS

Specific embodiments of the invention have been chosen for purposes of illustration and description, and are shown in the accompanying drawings, forming a part of the specification wherein:

FIG. 1 provides an isometric view of the tissue expansion barrier according to the invention;

FIG. 2 provides an isometric view of the inventive the tissue expansion barrier of further including pre-placed sutures penetrating said barrier;

FIG. 3 illustrates an elevation view of the tissue expansion barrier of the invention, further including pre-placed sutures penetrating said barrier;

FIG. 4 illustrates a second embodiment of the invention;

FIG. 5 provides a top elevation view of a second embodiment of the tissue expansion barrier of the invention;

FIG. 6 shows a further embodiment of the invention, having a trapezoidal cross section;

FIG. 7 shows a cross- section view of the tissue barrier of this invention as implanted according the inventive method; and

FIGS. 8a-d schematically illustrate implantation of the inventive device according to the inventive method

### DETAILED DESCRIPTION OF THE PREFERRED EMBODIMENTS

The present invention provides a new and enhanced apparatus and surgical method for the treatment of presbyopia. The tissue barrier implants (“implants”) and related implantation methodology have a number of advantages over prior art approaches, primarily through restoring accommodation by the contraction of the ciliary muscle in near vision situations. It should be understood that reference to a crystalline lens in the specification herein and the claims refers both to a natural lens and to an artificial intraocular lens in the phakic or pseudophakic eye, respectfully.

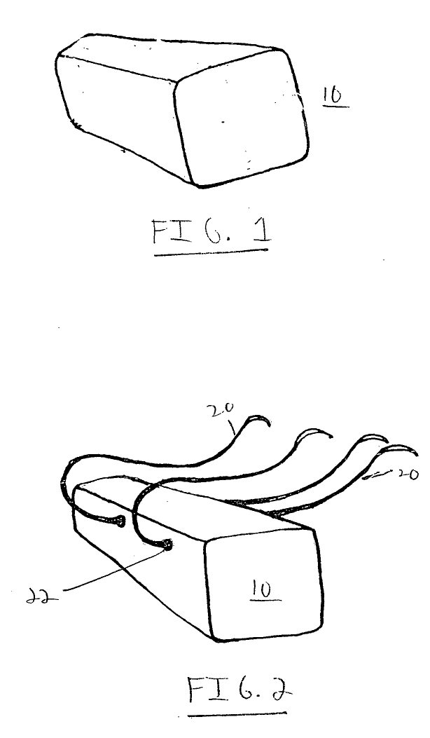

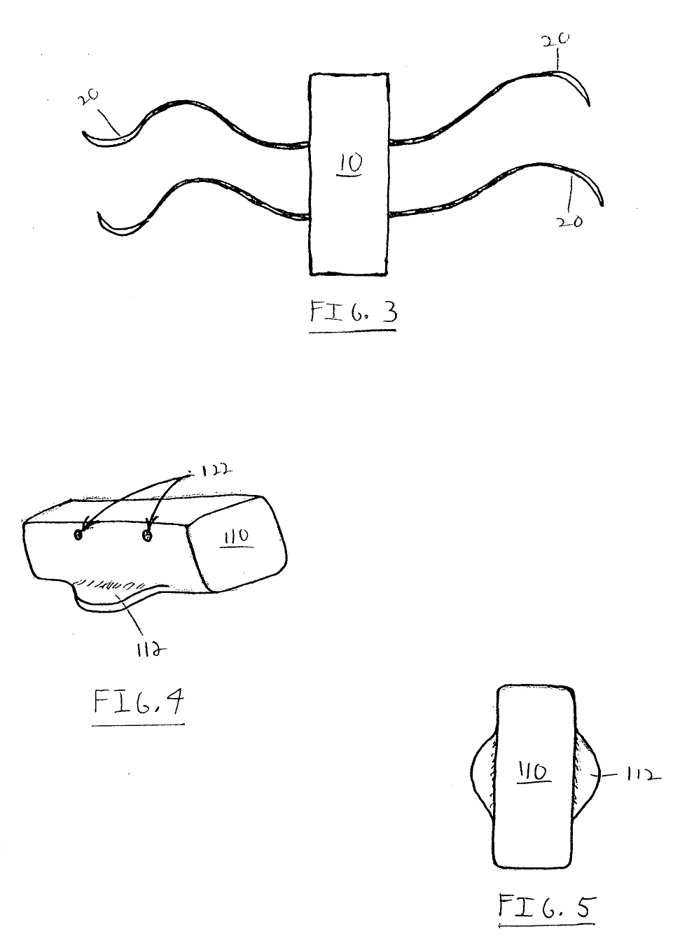

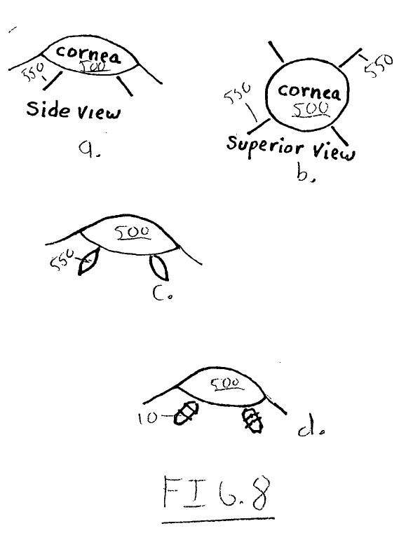

Referring now to FIGS. 1-3, the inventive scleral implant 10 consists of a specially designed rod or block of material such as silicone (in the preferred embodiment), titanium, polymethylmethacrylate, or other solid, biologically inert material that is placed into a radial incision made through the sclera over the ciliary muscle (ciliary body), and fixed in place (as further shown in FIGS. 7 and 8a-d). Fixation is achieved by sutures 20 that are pre-placed in suture holes 22 in the implant 10, preferably in the upper 25% of the barrier. Other means of fixation known in the art may also be used, such as a surgical adhesive. In the first embodiment of the invention, the inventive scleral implant 10 is generally formed in the shape of a rectangular block having rounded or chamfered corners.

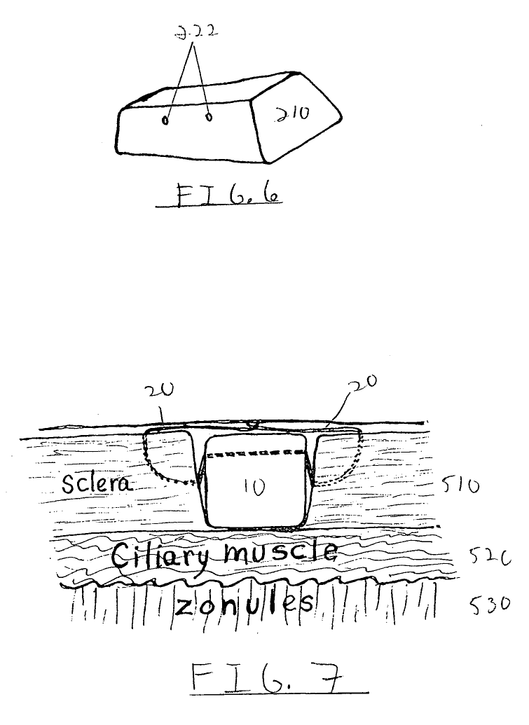

A second embodiment of the inventive implant 110 is illustrated in FIGS. 4 and 5. As with first embodiment of the invention, the implant 110 is generally shaped as a rectangular block having rounded or chamfered comers. Implant 110 additionally features outcroppings or wings 112 that assist in keeping the implant 110 in place and to keep the scleral incision open, according to the inventive method. As with the first embodiment, implant 110 is provided with suture holes 122 for accommodation of pre-placed sutures (not shown).

FIG. 6 shows a third embodiment of the inventive implant 210. As can be seen, implant 210 is trapezoidal in shape. Such shape further assists in keeping the implant in place and the scleral incision open. Suture holes 222 are provided, as is seen with the prior two embodiments.

Referring to FIGS. 7 and 8a-d, the inventive implant 10 (as well as embodiments 110 and 210) is unique in that it does not involve making circumferential tunnels or pockets in the sclera 510 as in most prior art nor are the implants 10 are placed circumferentially in tunnels or pockets in the sclera 510. It is not the purpose of the implant 10 to raise intraocular pressure nor is intraocular pressure intentionally raised. Quite to the contrary, eye pressure may be lowered, which is an additional feature of the invention. No external pressure is placed on the ciliary muscle 520 and no external force is applied on the zonules 530 by the implant 10. No attempt is made, nor is there any need for shortening of the zonules 530. The sclera 510 is not raised above the ciliary muscle 520. Expansion of the globe (comprising the ciliary muscles 520 and the zonules 530) is circumferential over the ciliary body 520 and is the result of stretching of the walls of the incision 550 and the natural force of the intraocular pressure on the relaxed globe wall. Unless closed by tight sutures or scar contraction, all incisions act as if tissue is added. The effect of added tissue is at right angles to the direction of the incision.

When radial incisions 550 are placed in the oblique quadrants of the sclera 510, beginning at the limbus and preferably carried 3 mm outward over the ciliary body, the scleral circumference expands (just as the cornea does in RK), producing an increase in the volume (an expansion) of the posterior chamber of the eye. The posterior chamber includes the ciliary muscle 520, the zonules (supporting fibrils connecting the ciliary muscle to the lens, making up the suspensory ligament of the lens) 530 and the crystalline lens (not shown). The incisions 550 are spread with spreading forceps and the tissue barriers 10 (or other embodiment thereof) are inserted and fixed with the pre-placed sutures to maintain expansion. With increased working space the lens/zonule complex can move forward with accommodative effort, correcting the loss of this function from presbyopia.

The means of relaxing the sclera according to the invention is by surgical incisions 550 but may also include chemical or laser or thermal application. According to the invention, use of the inventive implant 10 (and other embodiments thereof) will retain expansion of the globe in the area of the ciliary muscle (ciliary body) will result in increased working space in the ciliary muscle-zonule-lens complex and allow restored accommodation. Addition of the tissue barrier or retainer 10 maintains the expansion of the globe produced by radial incisions 550 over the ciliary body 520 and results in restoration of accommodation. Glaucoma and other conditions can also be treated by increasing the scleral circumference according to the invention. Pursuant to the invention, eye pressure is not increased but, to the contrary, the eye pressure may be lowered, which is another feature of the inventive method.

Further to understanding the importance of the inventive apparatus is the fact that, though the radial incisions placed over the ciliary body produce a restoration of the relationships of the structures of the posterior chamber as in a younger eye, the rapid healing and contraction of the walls of the incisions from neovascularization and scarring cause a loss of the effect quite rapidly, with a regression of effect occurring within a few weeks. An expansion barrier, such as barrier 10 prevents that contraction.

In addition to the structures, sequences, and uses immediately described above, it will be apparent to those skilled in the art that other modifications and variations can be made the method of the instant invention without diverging from the scope, spirit, or teaching of the invention. Therefore, it is the intention of the inventors that the description of instant invention should be considered illustrative and the invention is to be limited only as specified in the claims and equivalents thereto.

We claim:

1. A method of increasing the amplitude of accommodation of a human eye having a crystalline lens having an equator thereto, a ciliary muscle having a posterior chamber and suspensory zonule fibers, the method comprising increasing the effective working distance of the ciliary muscle from the lens by increasing the radial distance between the equator of the crystalline lens and the inner diameter of the ciliary muscle by enlarging the posterior chamber through intervention with external means.

2. The method of claim 1 wherein said radial distance is increased by circumferentially expanding the sclera adjacent to the ciliary body.

3. The method of claim 2 wherein the scleral expansion is accomplished by making radial incisions in the sclera, extending about 3 mm from the limbus over the ciliary body.

4. The method of claim 3 wherein said scleral expansion is further accomplished by stretching the radial incision.

5. The method of claim 4 wherein the stretching and expansion of the radial incision is maintained and preserved by insertion of a tissue barrier into the radial incision.

6. The method of claim 5 wherein said tissue barrier has an elongate body comprised of a biologically inert material and is adapted for insertion into an incision in the sclera of the eye to prevent tissue in-growth and maintain expansion.

7. A tissue barrier having an elongate body and comprised of a biologically inert material is adapted for insertion into an incision in the sclera of the eye to prevent tissue in-growth and maintain expansion of said incision

8. The tissue barrier of claim 7 wherein the barrier comprised silicone, whereby the barrier has the characteristics of being solid, flexible, inert, relatively soft, and similar in texture and appearance to the sclera.

9. The tissue barrier of claim 7 wherein said barrier is approximately 2.5 mm in length and approximately 0.6 mm in diameter.

10. The tissue barrier of claim 7 further comprising at least one integral pre-placed suture through the barrier wherein the barrier is fastened within the incision by said at least one integral pre-placed suture.

11. The tissue barrier of claim 10 further comprising an upper side, wherein the at least one integral pre-placed suture is located approximately ¼ the distance from the upper side.

12. The tissue barrier of claim 7 wherein the barrier is made of a material selected from the group consisting of titanium or polymethylmethacrylate.

13. The tissue barrier of claim 7 wherein the said tissue barrier is fastened to the incision by an adhesive.

14. The tissue barrier of claim 7 wherein the tissue barrier further comprises wings or projections extending from the sides or ends of the barrier.

15. The tissue barrier of claim 7 wherein the tissue barrier has a trapezoidal cross section.

16. A method of treating eye diseases selected from the group consisting of ocular hypertension and primary open angle glaucoma in a human eye having a crystalline lens having an equator thereto, a ciliary muscle and zonules connecting the ciliary muscle to the lens, the method comprising increasing the effective working distance of the ciliary muscle from the lens by increasing the radial distance between the equator of the crystalline lens and the inner diameter of the ciliary muscle by manipulating said muscle by intervention with external means

17. A method of treating presbyopia in a human eye having a crystalline lens, a sclera, a ciliary muscle and zonules connecting the ciliary muscle to the lens by increasing the effective working distance between the equator of the crystalline lens and the inner diameter of the ciliary muscle by increasing the radial distance between the equator of the crystalline lens and the inner diameter of the ciliary muscle by manipulating said muscle with intervention with external means.
